(19) United States
(12) Patent Application Publication
(10) Pub. No.: US 2026/0130621 A1
(43) Pub. Date: May 14, 2026

(54) SYSTEM AND METHOD TO SELECTIVELY DISPLAY MAPPING DATA BASED ON ELECTRODE ORIENTATION RELATIVE TO ADJACENT TISSUE

(71) Applicant: St. Jude Medical, Cardiology Division, Inc., St. Paul, MN (US)

(72) Inventor: Don Curtis Deno, Andover, MN (US)

(21) Appl. No.: 19/105,218

(22) PCT Filed: Aug. 22, 2023
(86) PCT No.: PCT/US2023/030849

§ 371 Date: Feb. 20, 2025

Related U.S. Application Data
(60) Provisional application No. 63/400,145, filed on Aug. 23, 2022.

(51) Int. Cl. A61B 5/367; A61B 5/283; A61B 5/343

(52) U.S. Cl. CPC A61B 5/367; A61B 5/283; A61B 5/343

(57) ABSTRACT

A method of utilizing orientation of groups of electrodes to selectively display beat/depolarization event data includes observing beat/depolarization event data at a plurality of electrodes located in an electrode array and determining a position of each of the plurality of electrodes. The method further includes calculating an orientation of each sub-set of electrodes relative to adjacent tissue and then determining whether to display beat/depolarization event data collected by each sub-set of electrodes based on the orientation of each sub-set of electrodes relative to the adjacent tissue.

### CROSS-REFERENCE TO RELATED APPLICATION

This application claims the benefit of and priority to U.S. provisional application 63/400,145, titled “SYSTEM AND METHOD TO SELECTIVELY DISPLAY MAPPING DATA BASED ON ELECTRODE ORIENTATION RELATIVE TO ADJACENT TISSUE”, filed Aug. 23, 2022, the contents of which are incorporated by reference herein.

### TECHNICAL FIELD

The present invention relates generally to catheters, and in particular to catheters utilized to perform electrophysiological (EP) mapping.

### BACKGROUND

Electrophysiology (EP) catheters are used in a variety of diagnostic, therapeutic, and/or mapping and ablative procedures to diagnose and/or correct conditions such as atrial or ventricular arrhythmias, including for example, ectopic atrial tachycardia, atrial fibrillation, and atrial flutter. Arrhythmias can create a variety of conditions including irregular heart rates, loss of synchronous atrioventricular contractions and stasis of blood flow in a chamber of a heart which can lead to a variety of symptomatic and asymptomatic ailments and even death.

Typically, a catheter is deployed and manipulated through a patient's vasculature to the intended site, for example, a site within a patient's heart. The catheter carries one or more electrodes that can be used for cardiac mapping or diagnosis, ablation and/or other therapy delivery modes, or both, for example. Once at the intended site, treatment can include, for example, radio frequency (RF) ablation, cryoablation, laser ablation, chemical ablation, high-intensity focused ultrasound-based ablation, microwave ablation, and/or other ablation treatments. The catheter imparts ablative energy to cardiac tissue to create one or more lesions in the cardiac tissue. To position a catheter at a desired site within the body, some type of navigation may be used, such as using mechanical steering features incorporated into the catheter (or a sheath). In some examples, medical personnel may manually manipulate and/or operate the catheter using the mechanical steering features.

Various catheter designs, such as for example, spline-based catheters with an array of electrodes, can be used to perform voltage mapping relative to the cardiac system as noted above. Voltage mapping is an important clinical tool to evaluate arrhythmogenic myocardium and guides further diagnostic and therapeutic procedures. In some embodiments, voltage data is collected from sub-sets of electrodes (e.g., 2, 3, 4, or more electrodes) organized into cliques. Voltage data monitored at a plurality of electrodes allows for wavefront attributes (e.g., direction, speed) to be determined for observed electrocardiogram signals (e.g., detected beats/depolarizations. The wavefront attributes can be projected onto adjacent cardiac tissue, allowing operators to visualize how electrical signals traverse cardiac tissue. However, mis-alignment between the electrodes observing the electrocardiogram signals and the tissue may result in erroneous projections of the wavefront attributes onto the tissue and displayed to an operator.

It would therefore be beneficial to provide a system and method of detecting mis-alignments between the orientation of the electrodes and the tissue to prevent erroneous mapping of EGM signals.

### SUMMARY

According to one aspect, a method of utilizing orientation of groups of electrodes to selectively display beat/depolarization event data includes observing beat/depolarization event data at a plurality of electrodes located in an electrode array and determining a position of each of the plurality of electrodes. The method further includes calculating an orientation of each sub-set of electrodes relative to adjacent tissue and then determining whether to display beat/depolarization event data collected by each sub-set of electrodes based on the orientation of each sub-set of electrodes relative to the adjacent tissue.

According to another aspect, a system for mapping cardiac electrophysiology information using a plurality of electrodes includes a processing apparatus configured to receive cardiac electrogram signals from the plurality of electrodes, receive location information for each of the plurality of electrodes, and calculate an orientation of each sub-set of electrodes relative to an orientation of adjacent tissue. The processing apparatus then determines whether to display electrophysiology information collected by each sub-set of electrodes based on the orientation of each sub-set of electrodes relative to the adjacent tissue.

### BRIEF DESCRIPTION OF THE DRAWINGS

FIG. 1A is a diagrammatic view of a system for generating surface models, mapping electrophysiological information thereon, and/or providing user interfaces, diagnostic information, electrophysiological vector representations, and positional information.

FIG. 1B is simplified diagrammatic and schematic view of the system illustrated in FIG. 1A.

FIG. 2A is an isometric view of an exemplary catheter having a plurality of electrodes organized into a grid-like structure according to some embodiments.

FIG. 2B is a diagrammatic view of a sub-set of electrodes included on the exemplary embodiment shown in FIG. 2A, organized into a triangular clique according to some embodiments.

FIG. 3 is a diagram illustrating visually the calculation of a surface normal vector and a clique normal vector according to some embodiments.

FIG. 4 is a flowchart illustrating a method utilized to determine whether observed beat/depolarization event data should be displayed, discarded, or hidden based on determined orientation of the electrodes relative to the adjacent tissue according some embodiments.

FIG. 5 is a flowchart illustrating in greater detail the utilization of clique normal vectors and surface normal vectors according to some embodiments.

### DETAILED DESCRIPTION

The present disclosure is directed to a system and method of determining orientation of sub-sets of electrodes relative to adjacent tissue. For example, in some embodiments the catheter utilized for electrophysiological (EP) mapping includes an array of electrodes. Information about the direction and speed of electrocardiogram signals propagating through cardiac tissue can be determined by monitoring voltages at a sub-set of electrodes (referred to as cliques). The reconstructed or visualized electrocardiogram signal is most accurate when the sub-set of electrodes are positioned immediately adjacent to the cardiac tissue being monitored. However, electrocardiogram signals are detected and visually projected onto the adjacent tissue regardless of contact with the tissue. If the orientation of the electrodes is not aligned or en face with respect to the adjacent tissue, the visual projection of the electrocardiogram signal onto the tissue may be erroneous. By determining the orientation of the electrodes relative to the adjacent tissue, a determination can be made regarding the accuracy of projecting the monitored electrocardiogram signal onto the adjacent tissue. In some embodiments, if the orientation of the electrodes (or sub-sets of electrodes) is sufficiently mis-aligned, the electrocardiogram signal observed by the sensors may be hidden or discarded. If the orientation of the sensors is sufficiently aligned, then the electrocardiogram signal is displayed normally to an operator.

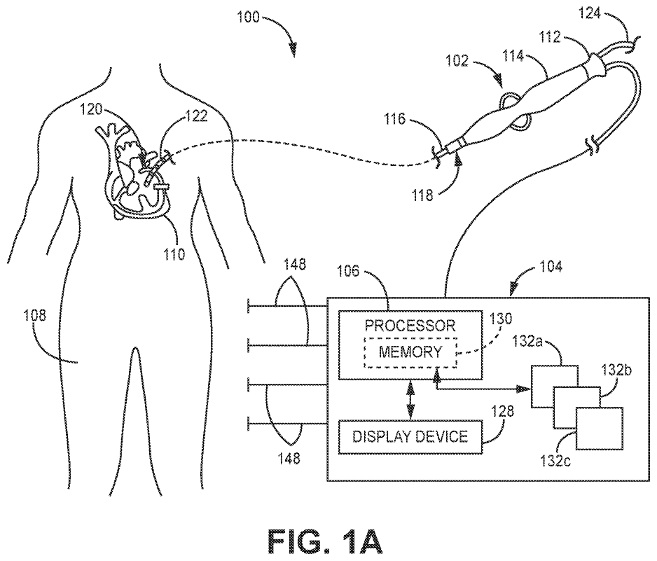

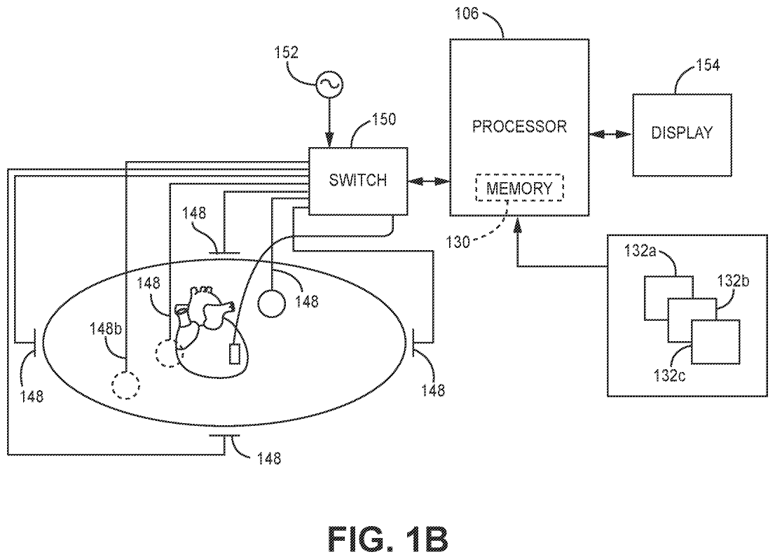

FIG. 1A illustrates one embodiment of a system 100 for mapping EP information corresponding to an anatomic structure onto a multi-dimensional (e.g., three-dimensional) geometry surface model (GSM) of the anatomic structure. The system 100 comprises, among other components, a medical device 102 and a data collection and analysis systems 104 suitable for collecting EP data and other data as described herein from a subject and to generate outputs that include data displays, user interfaces, and other OT related features disclosed herein. In one embodiment, the medical device 102 is a catheter, which includes cable connector or interface 112, a handle 114, a shaft 116 having a proximal end 118 and a distal end 120, and cables 124, 126. A plurality of electrodes 122 are located at the distal end 120 of the shaft 116 and may be utilized for a variety of functions, including impedance-based localization, EP mapping, and/or ablative treatments. The data collection and analysis system 104 includes a processing apparatus 106 having memory 130, and a display device 128. In some embodiments, the data collection and analysis system 104 is connected to a plurality of surface patch electrodes via connectors 148, which may be utilized to implement an impedance-based (or voltage-based) localization system for determining the location of the electrodes 122 within the patient's body 108 and/or heart 120. In addition, data collection and analysis systems 104 is configured to receive measurements observed by the catheter 102. For example, data collection and analysis system 104 may receive voltages monitored by the plurality of electrodes 122 located at the distal end of the catheter 102, both for impedance-based localization operations in conjunction with the surface patch electrodes 148 and electrocardiogram signals monitored by the plurality of electrodes 122.

The processing apparatus 106 may include one or more apparatus, devices, and machines for processing data, signals and information, including by way of example a programmable processor, a computing device such as a computer, or multiple processors or computers. The apparatus can include, in addition to hardware, code that creates an execution environment for the computer program in question, e.g., code that constitutes processor firmware, a stack, a data management system, an operating system, one or more user interface systems, or a combination of one or more of them. Further, the processing apparatus 106 can include machine readable medium or other memory that includes one or more software modules 132a, 132b, and 132c for performing various functions. In some embodiments, software modules 132a, 132b, and/or 132c receive voltages measured by the plurality of electrodes 122 related to beats/depolarizations within the hear 120. In some embodiments, electrocardiogram signals monitored by the plurality of electrodes 122 can be utilized to detect a beat/depolarization event, wherein analysis of signals monitored by a sub-set of electrodes (e.g., clique) can be utilized to determine wavefront attributes of the beat/depolarization event such as activation direction (AD) and wave speed (WS). In some embodiments, the processing apparatus 106 projects the determined wavefront attributes onto the geometry surface model (GSM) of the cardiac structure and displays the information to an operator via display device 128. This allows an operator to visualize the propagation of an electrical signal through the tissue of the heart 120. In some embodiments, system 104 may utilize omnipolar detection of electrocardiogram signals as described, for example, in U.S. Pat. No. 10,758,137 entitled “Orientation independent sensing, mapping, interface and analysis systems and methods”, the entire disclosure of which is incorporated herein by reference.

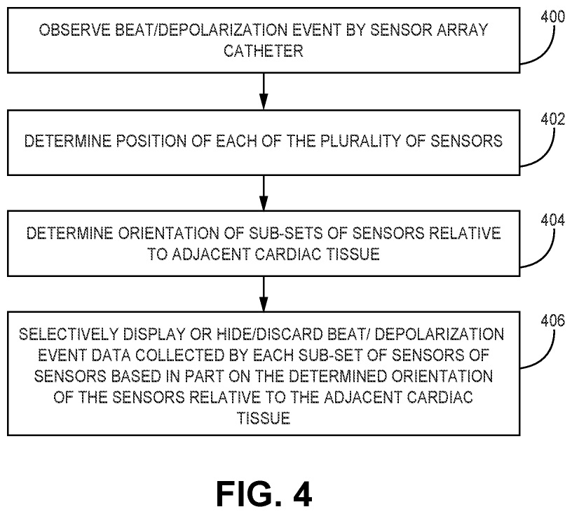

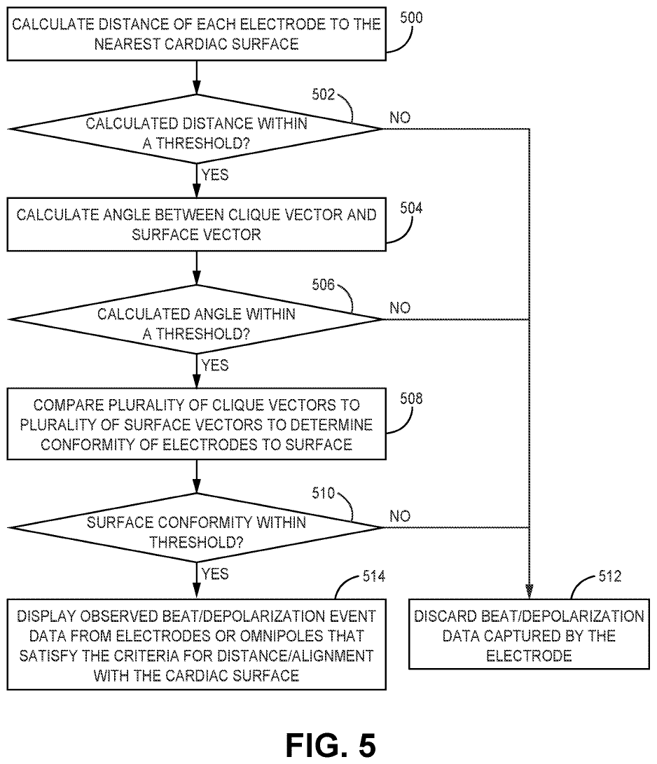

Analyzing electrocardiogram signals and projecting attributes of observed betas/depolarizations onto GSMs of the cardiac structure depends in part on the orientation of the sensors 122 relative to the cardiac structure being monitored. As described in more detail with respect to the methods shown in FIGS. 4 and 5, processing apparatus and corresponding software modules 132a, 132b, and 132c utilize location data associated with the plurality of electrodes to determine the orientation of sub-sets of electrodes relative to the orientation of the adjacent tissue. The orientation of the sub-sets of electrodes relative to adjacent tissue is utilized in determining whether to display wavefront attributes associated with observed beat/depolarization events. In particular, in some embodiments data provided by sub-sets of electrodes having an orientation mis-aligned with adjacent tissue may not be displayed to a user via display device 128.

With reference to FIG. 1B, components utilized in providing an impedance-based localization system are shown in additional detail. For the sake of clarity these elements were not shown in FIG. 1A but may be included. For example, in some embodiments the system may include, among other possible components, a plurality of patch electrodes 148, a multiplex switch 150, a signal generator 152, and a display device 154. In another exemplary embodiment, some or all of these components are separate and distinct from system 104 but that are electrically connected to, and configured for communication with, system 104.

In some embodiments, three pairs of patch electrodes 148 are provided, plus a reference patch electrode 148b located on a patient's belly. The pairs of patch electrodes are typically attached on opposite sides of the patient's body. A high-frequency signal generated by signal generator 152 is selectively applied between pairs of patches 148 by switching circuit 150 to create an electric field across the patient's body 108. The electrodes 122 located on the catheter measure the high-frequency signal and provide the monitored voltage to the processing apparatus 106, which utilizes the monitored voltage (or impedance) to determine the location of each electrode 122. In some embodiments, system 104 may utilize the type of electric field-based (impedance-based) system as that utilized in the EnSite™ NavX system commercially available from Abbott Laboratories, and generally shown with reference to U.S. Pat. No. 7,263,397 entitled “Method and Apparatus for Catheter Navigation and Location and Mapping in the Heart”, the entire disclosure of which is incorporated herein by reference. Another exemplary system 104 is the EnSite Precision™ system, which uses both electric field-based (i.e., impedance based) and magnetic based localization to determine the location of sensors, including electrodes 122. Location for each electrode 122 is important in order to determine the orientation of sub-sets of electrodes 122 relative to adjacent tissue. In other embodiments, however, various other methods may be utilized either alone or in conjunction with the method described with respect to FIG. 1B for determining the location of the electrodes 122 within the patient's body.

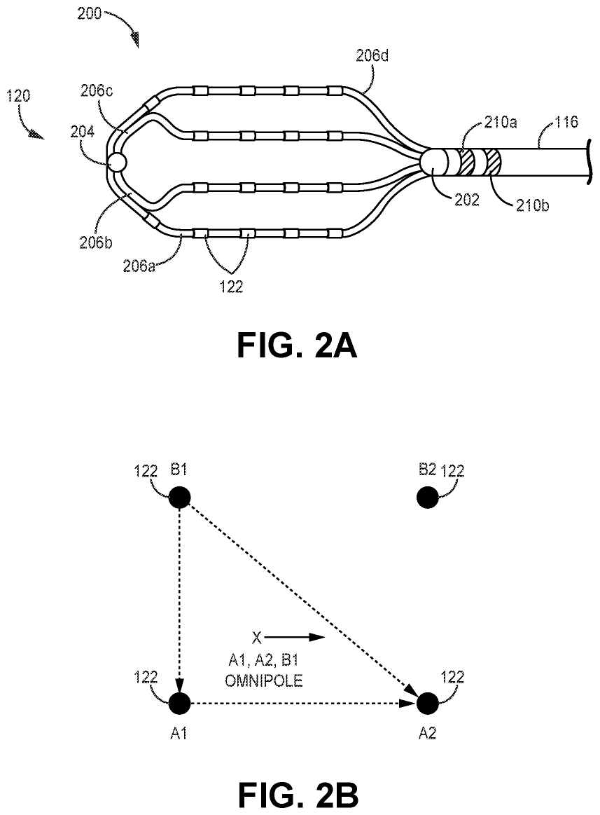

FIG. 2A is a side view of an electrode array or paddle 200 located at the distal end 120 of the catheter 116 according to some embodiments. As shown in FIG. 2A, the electrode array or paddle 200 includes a proximal end 202 and a distal end 204. A plurality of arms 206a, 206b, 206c, and 206d are connected between the proximal end 202 and the distal end 204. Each of the plurality of arms 206a, 206b, 206c, and 206d include a plurality of sensors or electrodes 122 spaced out along the length of the arms 206a, 206b, 206c, and 206d. In the embodiment shown in FIG. 2A each of the plurality of arms 206a, 206b, 206c, and 206d include four electrodes 122. In other embodiments, the paddle 200 may include fewer or additional arms and each arm may include fewer or additional electrodes. In some embodiments, additional electrodes 210a, 210b are located proximal of the paddle 200.

Electrodes 122 are utilized to sense EGM signals resulting from cardiac beat/depolarization events. Processing apparatus 106 utilizes the acquired EGM signals to visualize how beat/depolarization wavefronts move along the cardiac surface, which can be useful in identifying conditions such as arrhythmias. In some embodiments, timing and monitored voltages are utilized to visualize the wavefront (i.e., cardiac depolarization) along the cardiac surface. In some embodiments, the waveshape of detected beat/depolarizations events are utilized in combination with unipolar and/or bipolar voltages monitored by sub-sets of electrodes 122 (referred to as a clique) to visualize the wavefront. For example, with reference to FIG. 2B, a plurality of electrodes 122a1, 122a2, 122b1, and 122b2 are shown. Electrodes 122al and 122a2 are located on a first arm 206a and electrodes 122b1 and 122b2 are located on a second arm 206b. In some embodiments the distance between electrode 122a1 and 122a2 is approximately equal to the distance between electrode 122a1 and 122b1. In the embodiment shown in FIG. 2B, a first clique is comprised of electrodes 122a1, 122a2, and 122b1. In some embodiments, bipolar voltages defined between electrodes 122b1 and 122al, between electrodes 122b1 and 122a2, and between electrodes 122al and 122a2 are measured and utilized to define an omnipolar (identified by the ‘x’ labeled A1, A2, B1 omnipole). In some embodiments, the plurality of bipole voltages are utilized generate wavefront attributes positioned at location approximately in the middle of the triangle. In some embodiments, wavefront attributes may include activation direction (AD), velocity or wavespeed (WS) of the corresponding depolarization, and/or maximum voltages associated with measured voltages (Vmax). In some embodiments, omnipolar attributes (e.g., AD, WS, and/or Vmax) are defined for each clique of electrodes. In the embodiments shown in FIG. 2B, defining each clique as a grouping of three electrodes allows for four individual cliques and four corresponding omnipoles to be defined. The first omnipole is defined by electrodes 122a1, 122a2, and 122b1, the second omnipole is defined by electrodes 122a1, 122a2, and 122b2, the third ominipole is defined by electrodes 122a2, 122b1, and 122b2, and the fourth omnipole is defined by electrodes 122a1, 122b1, and 122b2.

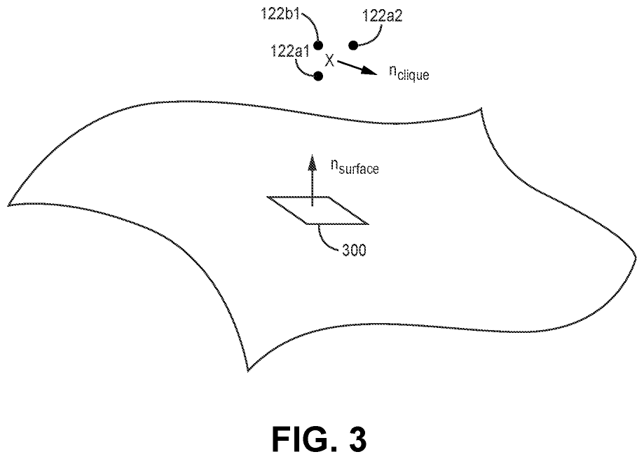

In some embodiments, the omnipole attributes including one or more of AD, WS and/or Vmax are two-dimensional (2D) attributes that are projected onto the cardiac surface being mapped. Projections are more accurate if the electrodes utilized to collect the EGM signal are facing (i.e., en face) with the cardiac tissue onto which the wavefront attributes (e.g., activation direction, wavespeed, etc.) are projected. This problem is illustrated in FIG. 3, which illustrates a plurality of electrodes 122a1, 122a2, and 122b1, mis-aligned with the nearest cardiac surface 300. In some embodiments, wavefront attributes calculated with respect to the plane defined by a clique of electrodes (e.g., 122a1, 122a2, and 122b1) projected onto the cardiac surface 300 may result in large errors in the calculated direction and velocity of a wavefront. For example, in some embodiments errors in direction of wavefront may approach 90°, with said error in direction resulting in a corresponding error in the speed of the wavefront. To prevent erroneous wavefront calculations from being relied upon as accurate, the orientation of one or more of a plurality of cliques is calculated and compared to the orientation of the cardiac surface onto which the wavefront is attributes are projected. In some embodiments, clique orientation is utilized in conjunction with electrode 122 and/or clique distance from the adjacent cardiac tissue 300.

FIG. 4 is a flowchart illustrating steps utilized by processing apparatus 106 in determining whether to display EGM data obtained from the electrodes 122. Reference is made to apparatus described with respect to FIG. 1A-3, although in other embodiments other apparatus and systems may be utilized to implement the method described herein.

At step 400, the electrodes 122 located on the electrode array or paddle 200 observe a beat/depolarization event. In some embodiments, this may include measuring the voltage at each of the plurality of electrodes or electrodes for a duration of time corresponding with the beat. In some embodiments, this includes measuring unipolar and/or bipolar voltages and utilizing the measured voltages to determine wavefront attributes associated with the observed beat/depolarization event, such as activation direction (AD) and wave speed (WS). As described with respect to FIG. 2B, in some embodiments a clique of electrodes (e.g., electrodes 122a1, 122a2, 122b1) are utilized to calculate an omnipole that characterizes attributes such as AD and WS. Wavefront attributes associated with a beat/depolarization event can then be projected onto the adjacent (i.e., closest) cardiac surface and displayed to an operator. Steps 402, 404, and 406 act to filter or determine whether the orientation of the electrodes observing a beat/depolarization event are mis-aligned with the cardiac surface onto which the wavefront attributes are projected. If a mis-alignment is detected, then wavefront attributes associated with the observed beat/depolarization event may include errors, in which case a determination may be made to hide or discard the collected data.

At step 402, the position of each of the plurality of electrodes 122 utilized to observe the beat/depolarization event is determined. In some embodiments, electrode position is determined using impedance localization techniques described with respect to FIG. 1B, in which electric fields created between respective surface patches are utilized to determine the position of individual electrodes 122 located on the paddle 200. In other embodiments, other means of determining the position of the electrodes 122 may be utilized, including magnetic-based localization technique and/or combinations of impedance-based localization techniques and magnetic-based localization techniques (as well as other systems and methods of determining the location of the electrodes).

At step 404, the orientation of sub-sets of electrodes are determined relative to the orientation of adjacent tissue (i.e., tissue closest to each sub-set of electrodes). As described above, in some embodiments each sub-set of electrodes are referred to as a clique. In some embodiments a clique may be comprised of a single electrode 122, but in general a clique of electrodes 122 includes at least three electrodes (as shown in FIG. 2B). A particular electrode 122 may be included as part of a plurality of different cliques. That is, an electrode may be a part of both a first and second clique (i.e., sub-set of electrodes).

Based on the locations of each of the three electrodes (e.g., 122a1, 122a2, and 122b1), a 2D plane is defined, which describes the orientation of each particular sub-set or clique of electrodes 122. The orientation of a particular sub-set or clique of electrodes 122 is compared to the orientation of adjacent tissue. Typically, the adjacent tissue represents the tissue closest to the sub-set of electrodes 122 and corresponds with the tissue onto which wavefront attributes (e.g., AD, WS, etc.) are projected. As described above, mis-alignment in the orientation of sub-set of electrodes 122 relative to the adjacent tissue may result in the erroneous projection of wavefront attributes onto the tissue. Determining the orientation of each of the sub-sets of electrodes relative to the tissue may be performed in a number of ways. For example, as described in more detail with respect to FIG. 5, a vector oriented normal to the plane defining the electrodes may be calculated and compared to a vector oriented normal to the adjacent tissue. Alignment between the respective vectors indicates that the orientation of the sub-set of electrodes 122 are aligned with the tissue 300. Increases in the difference between the respective vectors indicates a mis-alignment in the orientation of the sub-set of electrodes 122 relative to the tissue 300. In some embodiments the orientation of each sub-set of electrodes 122 (i.e., each clique) is compared to the orientation of adjacent tissue and decisions of whether to include or discard data is made by on a clique-by-clique basis. One of the benefits of making a determination on a clique-by-clique basis is that some useful data can be extracted even if portions of the paddle 200 are mis-aligned or not close enough to the cardiac tissue. In other embodiments, the orientation of a plurality of sub-sets of electrodes 122 are utilized in combination to determine whether the electrode array—as a whole—conforms to the geometry of the tissue surface. In some embodiments, this comparison may result in discarding all data collected by the electrodes if the electrode array or paddle 200 is found to be generally not aligned or lacking in conformity with the orientation of the surface.

At step 406, a determination is made whether to utilize observed beat/depolarization event data based, at least in part, on the determined orientation of the electrodes 122 relative to the adjacent tissue 300. In some embodiments, the determination of whether to utilize observed beat/depolarization event data is also based on criteria utilized in combination with determined orientation relative to the tissue, including but not limited to the determined distance of each of the plurality of electrodes 122 to the cardiac tissue 300. In some embodiments, each sub-set of electrodes 122 is analyzed individually to determine whether to display or discard/hide beat/depolarization data. For example, a first sub-set of electrode 122 may be oriented en face relative to the adjacent tissue and beat/depolarization data captured by the first sub-set of electrode 122 would be utilized, while a second set of electrodes 122 may have an orientation offset from the adjacent tissue to preclude utilization of beat/depolarization data captured by the second sub-set of electrode 122. As described in more detail in FIG. 5, in some embodiments the difference in orientation determined between each sub-set of electrodes 122 and the adjacent cardiac tissue 300 is compared to a threshold value to determine whether the sub-set of electrodes 122 is sufficiently en face. In some embodiments, the threshold is predetermined. In other embodiments the threshold may be selected or modified by the operator. As described in more detail, defining normal vectors extending from the plane defining the sub-set of electrodes 122 and a normal vector extending from an adjacent tissue surface allows the orientation of the sub-set of electrodes to be determined relative to the surface by finding the angle or difference between the respective normal vectors. This difference between the respective vectors is less than a threshold value then the sub-set of electrodes 122 is determined to be a sufficiently en face to merit displaying the beat/depolarization data captured by the sub-set of electrodes 122. If the difference is larger than the threshold, then the sub-set of electrode 122 are determined to be sufficiently mis-aligned with the surface, wherein the beat/depolarization data is discarded or hidden from view.

In some embodiments, observed beat/depolarization data that is not to be utilized is discarded (i.e., deleted). Discarding of the observed beat/depolarization event data prevents the data from being stored or viewed. In some embodiments, observed beat/depolarization data that is not be utilized is hidden rather than deleted. Hiding of the observed beat/depolarization event data may allow the data to be subsequently viewed. For example, in some embodiments an operator may modify the thresholds utilized to determine whether the observed beat/depolarization event data observed by a particular clique is utilized or not. In the event the threshold is increased, observed beat/depolarization event data that was previously hidden may be displayed to an operator. In some embodiments, if different thresholds may be utilized in determining whether to discard or hide observed beat/depolarization event data. That is, a first threshold may be utilized to determine whether observed beat/depolarization data should be discarded and a second threshold may be utilized to determine whether observed beat/depolarization data should be hidden rather than discarded. In this example, the second threshold would be less than the first threshold (second threshold would be indicative of better alignment than the first threshold). Observed beat/depolarization data collected by electrodes 122 properly aligned or oriented relative to the tissue as determined at step 406 are utilized, typically via display of wavefront attributes (e.g., AD, WS, etc.) associated with the observed beat/depolarization event. In still other embodiments, the threshold(s) may be determined via a form of machine learning that incorporates previous operator judgment. For example, machine learning may be initiated by placing a sub-set of electrodes in and unambiguously en face position as well as in unambiguously mis-aligned positions and generated feedback that is reviewed by a human operator (without knowledge of which data is obtained by en face electrodes and mis-aligned electrodes). The operator would rate the data obtained by the respective electrodes as trustworthy or not based on their experience. Logistical regression analysis may then be utilized to with previously identified variables of distance, angular difference, and surface curvature conformity alone or in combination to determine thresholds. These may be identified as initial or default thresholds, with operators able to adjust the thresholds up or down as desired. In other embodiments, other means of selecting the thresholds may be utilized, including artificial training based on learning models that include examples of both sub-sets of electrodes oriented en face to the adjacent tissue and mis-aligned with the adjacent tissue.

FIG. 5 is a flowchart illustrating in additional detail steps utilized by processing apparatus 106 in determining whether to display EGM data obtained from the electrodes 122. At step 500, the distance of each electrode 122 to the nearest cardiac surface is calculated based on location information obtained with respect to each electrode. At step 502 the calculated distance is compared to a threshold value. If the calculated distance is not within a threshold value (i.e., electrode 122 is located too far from the nearest cardiac surface) then at step 512 beat/depolarization data captured by that electrode is discarded. In some embodiments, this includes discarding not only beat/depolarization data by the particular electrode 122, but also discarding beat/depolarization data captured by a clique that includes the particular electrode 122. If the calculated distance is within the threshold value, then the process proceeds to step 504.

At step 504, the angle or difference in orientation between clique normal vectors and surface normal vectors are calculated. This requires that the orientation of the clique normal vectors and surface normal vectors are calculated or known. In some embodiments, clique normal vectors are calculated for the plurality of cliques included within an electrode array based on the determined location of the plurality of electrodes 122. Generally, the same location of the electrodes utilized to determine distance of the electrodes 122 from the cardiac surface 300 is utilized to determine the orientation of the electrodes 122 relative to the cardiac surface 300. In some embodiments, clique normal vectors are calculated only for those cliques comprised of electrodes located within the threshold distance to the cardiac surface as determined at steps 500 and 502. In other embodiments, clique normal vectors are calculated for each clique regardless of distance of electrodes making up the clique. Likewise, surface normal vectors are calculated (or in some embodiments, already known) for surfaces located adjacent or at least closest to the electrode array.

In some embodiments, the angle or difference in orientation between a clique normal vector and an adjacent surface normal vector is determined by calculating the dot product of the respective vectors according to the equation:

$$\frac{\vec{a} \cdot \vec{b}}{\vert a \vert \vert b \vert} = cos θ \qquad Eq . 1$$

wherein θ is the difference in the vectors \vec{a} and \vec{b}. In some embodiments, the vectors \vec{a} and \vec{b} are unit vectors so that the denominator of the term is equal to one.

At step 506, a determination is made whether the calculated angle θ between the orientation of the clique normal vector and the adjacent surface normal vector is within a threshold (i.e., whether the clique is sufficiently aligned or en face with the cardiac surface). If the calculated angle is greater than the threshold, indicating that the plane defining the clique of electrodes 122 is not oriented or aligned with the adjacent cardiac surface 300, then at step 512 the beat/depolarization data captured by the electrodes 122 associated with the clique are discarded/hidden. If the calculated angle is less than the threshold, indicating that the plane defining the clique of electrodes 122 is oriented or aligned with the adjacent cardiac surface 300, then the process continues at step 508. In some embodiments, steps 508 and 510 are utilized to provide an additional comparison of a plurality of cliques to determine whether the electrode array or paddle is aligned or conforms with the cardiac surface 300. In other embodiments, the comparison performed at steps 506 and 508 is sufficient.

At step 508, a plurality of clique normal vectors are compared to a plurality of surface normal vectors. In some embodiments, the comparison may include comparing the change in clique normal vector orientations between adjacent cliques to the change in surface normal vectors between adjacent cardiac surfaces. For example, if a difference in orientation between a first clique normal vector and an adjacent clique normal vector is equal to 10°, this change or bend in orientation of the electrode array or paddle 200 can be compared to corresponding differences between adjacent surface normal vectors. For example, if the difference between corresponding first and second surface normal vectors is also 10° (or within a threshold difference) then that is compelling evidence that the electrode array is bent or conformed to the cardiac surface. In contrast, if the difference in orientation between the first clique normal vector and an adjacent surface normal vector is 0° and the difference in orientation between corresponding first and second surface normal vectors is 20°, this is an indication of a lack of conformity between the respective cliques and the cardiac surface, which indicates that the respective cliques are likely not in contact with the corresponding cardiac surfaces. In other embodiments, other methods may be utilized to compare or measure the degree of conformity between a plurality of clique vectors and the surface normal vectors. For example, principles of Gaussian curvature methods may be utilized to determine the orientation and amount of local curvature associated with each of the plurality of cliques as well as the orientation and amount of local curvature associated with adjacent tissue based on anatomic models generated by the 3D mapping system. In some embodiments, mean principle curvature (e.g., arithmetic mean of the curvatures k) and principal curvatures representing the minimum and maximum values of the curvature are utilized to compare the curvature of the plurality of electrodes to the adjacent tissue surface. In other embodiments, other means of comparing the respective surface curvatures could be utilized. A lack of conformity between the plurality of electrodes and the adjacent tissue surface would indicate, for example with respect to the grid of electrodes located on the paddle, that the paddle is flat and does not conform to the nearby curved tissue surface.

In some embodiments, if at step 510 it is determined that there is a lack of conformity between the plurality of electrodes and the adjacent tissue surface, then at step 512 the beat/depolarization data captured by the cliques are discarded and/or hidden. In some embodiments, this step prevents erroneous estimates of Vmax, AD, or WS from being displayed to the user in situations in which the electrodes are likely not located adjacent to the tissue surface.

If at step 510 the surface conformity is within the threshold then at step 514 the beat/depolarization data captured by the cliques are displayed to the user and/or saved. In this embodiment, a combination of distance, orientation and conformity is utilized to determine whether data collected by the electrodes is displayed to the operator and/or saved. In some embodiments, data collected by a subset of electrodes may be hidden/discarded and data collected by another subset of electrodes may be utilized or displayed to the operator. That is, criteria may be applied on an electrode-by-electrode basis, a clique-by-clique basis, or to the entire electrode array or paddle 200.

### DISCUSSION OF POSSIBLE EMBODIMENTS

The following are non-exclusive descriptions of possible embodiments of the present invention.

According to one aspect, a method of utilizing orientation of groups of electrodes to selectively display beat/depolarization event data includes observing beat/depolarization event data at a plurality of electrodes located in an electrode array and determining a position of each of the plurality of electrodes. The method further includes calculating an orientation of each sub-set of electrodes relative to adjacent tissue and then determining whether to display beat/depolarization event data collected by each sub-set of electrodes based on the orientation of each sub-set of electrodes relative to the adjacent tissue.

The method of the preceding paragraph may optionally include, additionally and/or alternatively, any one or more of the following features, configurations and/or additional components.

For example, in some aspects the step of calculating an orientation of each sub-set of electrodes relative to an orientation of adjacent tissue further includes defining a plane that extends through each electrode included in the sub-set of electrodes, calculating an electrode orientation vector normal to the defined plane, calculating a surface vector normal to the adjacent tissue, and calculating a difference between the electrode orientation vector and the surface vector.

In some embodiments, determining whether to display beat/depolarization event data may be based on the calculated difference between the electrode orientation vector and the surface vector.

In some embodiments, calculating a difference between the electrode orientation vector and the surface vector may further include calculating a dot product of the electrode orientation vector and the surface vector.

In some embodiments, determining whether to display beat/depolarization event data may include comparing the calculated difference to a threshold value.

In some embodiments, the method may further include calculating a distance from each sensor to the adjacent tissue, wherein determining whether to display beat/depolarization event data collected by each sub-set of sensors is based, at least in part, on the calculated distance from each sensor to the adjacent tissue.

In some embodiments, the calculated distance may be compared to a threshold value to determine whether to display beat/polarization event data.

In some embodiments, the method may further include comparing orientations of a plurality of sub-set of electrodes to orientations of a plurality of cardiac surfaces to determine conformity between the electrode array and the cardiac surface.

In some embodiments, comparing orientations of a plurality of sub-set of electrodes to orientations of a plurality of cardiac surfaces includes comparing changes in orientation between adjacent sub-sets of electrodes to changes in orientation between adjacent cardiac surfaces.

In some embodiments, determining whether to display beat/depolarization event data collected by each sub-set of electrodes may be based further on the conformity of the plurality of sub-sets of electrodes to the plurality of cardiac surfaces.

According to another aspect, a system for mapping cardiac electrophysiology information using a plurality of electrodes organized into a plurality of sub-sets of electrodes includes a processing apparatus configured to receive cardiac electrogram signals from the plurality of electrodes, receive location information for each of the plurality of electrodes, calculate an orientation of each sub-set of electrodes relative to an orientation of adjacent tissue, and determine whether to display electrophysiology information collected by each sub-set of electrodes based on the orientation of each sub-set of electrodes relative to the adjacent tissue.

The system of the preceding paragraph may optionally include, additionally and/or alternatively, any one or more of the following features, configurations and/or additional components.

For example, in some embodiments the processing apparatus calculates the orientation of each sub-set of electrodes relative to the orientation of adjacent tissue by defining a plane that extends through each electrode included in the sub-set of electrodes, calculating an electrode orientation vector normal to the defined plane, calculating a surface vector normal to the adjacent tissue, and calculating a difference between the electrode orientation vector and the surface vector.

In some embodiments, the processing apparatus may determine whether to display electrophysiology information based on the calculated difference between the electrode orientation vector and the surface vector.

In some embodiments, the processing apparatus may calculate a difference between the electrode orientation vector and the surface vector by calculating a dot product of the electrode orientation vector and the surface vector.

In some embodiments, the processing apparatus may determine whether to display electrophysiology information by comparing the calculated difference to a threshold value.

In some embodiments, the processing apparatus is further configured to calculate a distance from each sensor to the adjacent tissue, wherein determining whether to display electrophysiology information collected by each sub-set of sensors may be based, at least in part, on the calculated distance from each sensor to the adjacent tissue.

In some embodiments, the processing apparatus may determine whether to display electrophysiology information by comparing the calculated distance to a threshold value.

In some embodiments, the processing apparatus may be further configured to compare orientations of a plurality of sub-set of electrodes to orientations of a plurality of cardiac surfaces to determine conformity between the plurality of sub-set of electrodes and the cardiac surface.

In some embodiments, the processing apparatus compares orientations of a plurality of sub-set of electrodes to orientations of a plurality of cardiac surfaces by comparing changes in orientation between adjacent sub-sets of electrodes to changes in orientation between adjacent cardiac surfaces.

In some embodiments, the processing apparatus determines whether to display electrophysiology information collected by each sub-set of electrodes based in addition on the conformity of the plurality of sub-sets of electrodes to the plurality of cardiac surfaces.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of utilizing orientation of groups of electrodes to selectively display beat/depolarization event data, the method comprising:
observing beat/depolarization event data at a plurality of electrodes located in an electrode array, wherein the plurality of electrodes are organized into a plurality of sub-sets of electrodes, each sub-set of electrodes including two or more electrodes;
determining a position of each of the plurality of electrodes;
calculating an orientation of each sub-set of electrodes relative to adjacent tissue; and
determining whether to display beat/depolarization event data collected by each sub-set of electrodes based on the orientation of each sub-set of electrodes relative to the adjacent tissue.

2. The method of claim 1, wherein calculating an orientation of each sub-set of electrodes relative to an orientation of adjacent tissue further includes:
defining a plane that extends through each electrode included in the sub-set of electrodes;
calculating an electrode orientation vector normal to the defined plane;
calculating a surface vector normal to the adjacent tissue; and
calculating a difference between the electrode orientation vector and the surface vector.

3. The method of claim 2, wherein determining whether to display beat/depolarization event data is based on the calculated difference between the electrode orientation vector and the surface vector.

4. The method of claim 2, wherein calculating a difference between the electrode orientation vector and the surface vector includes calculating a dot product of the electrode orientation vector and the surface vector.

5. The method of claim 3, wherein determining whether to display beat/depolarization event data includes comparing the calculated difference to a threshold value.

6. The method of claim 1, further including calculating a distance from each sensor to the adjacent tissue, wherein determining whether to display beat/depolarization event data collected by each sub-set of sensors is based, at least in part, on the calculated distance from each sensor to the adjacent tissue.

7. The method of claim 6, wherein the calculated distance is compared to a threshold value to determine whether to display beat/polarization event data.

8. The method of claim 7, further including comparing orientations of a plurality of sub-set of electrodes to orientations of a plurality of cardiac surfaces to determine conformity between the electrode array and the cardiac surface.

9. The method of claim 8, wherein comparing orientations of a plurality of sub-set of electrodes to orientations of a plurality of cardiac surfaces includes comparing changes in orientation between adjacent sub-sets of electrodes to changes in orientation between adjacent cardiac surfaces.

10. The method of claim 8, wherein determining whether to display beat/depolarization event data collected by each sub-set of electrodes further is based further on the conformity of the plurality of sub-sets of electrodes to the plurality of cardiac surfaces.

11. A system for mapping cardiac electrophysiology information using a plurality of electrodes organized into a plurality of sub-sets of electrodes, the system comprising:
a processing apparatus configured to:
receive cardiac electrogram signals from the plurality of electrodes;
receive location information for each of the plurality of electrodes;
calculate an orientation of each sub-set of electrodes relative to an orientation of adjacent tissue; and
determine whether to display electrophysiology information collected by each sub-set of electrodes based on the orientation of each sub-set of electrodes relative to the adjacent tissue.

12. The system of claim 11, wherein the processing apparatus calculates the orientation of each sub-set of electrodes relative to the orientation of adjacent tissue by:
defining a plane that extends through each electrode included in the sub-set of electrodes;
calculating an electrode orientation vector normal to the defined plane;
calculating a surface vector normal to the adjacent tissue; and
calculating a difference between the electrode orientation vector and the surface vector.

13. The system of claim 12, wherein the processing apparatus determines whether to display electrophysiology information based on the calculated difference between the electrode orientation vector and the surface vector.

14. The system of claim 12, wherein the processing apparatus calculates a difference between the electrode orientation vector and the surface vector by calculating a dot product of the electrode orientation vector and the surface vector.

15. The system of claim 12, wherein the processing apparatus determines whether to display electrophysiology information by comparing the calculated difference to a threshold value.

16. The system of claim 11, wherein the processing apparatus is further configured to:
calculate a distance from each sensor to the adjacent tissue, wherein determining whether to display electrophysiology information collected by each sub-set of sensors is based, at least in part, on the calculated distance from each sensor to the adjacent tissue.

17. The system of claim 16, wherein the processing apparatus determines whether to display electrophysiology information by comparing the calculated distance to a threshold value.

18. The system of claim 17, wherein the processing apparatus is further configured to:
compare orientations of a plurality of sub-set of electrodes to orientations of a plurality of cardiac surfaces to determine conformity between the plurality of sub-set of electrodes and the cardiac surface.

19. The system of claim 18, wherein the processing apparatus compares orientations of a plurality of sub-set of electrodes to orientations of a plurality of cardiac surfaces by comparing changes in orientation between adjacent sub-sets of electrodes to changes in orientation between adjacent cardiac surfaces.

20. The system of claim 18, wherein the processing apparatus determines whether to display electrophysiology information collected by each sub-set of electrodes based in addition on the conformity of the plurality of sub-sets of electrodes to the plurality of cardiac surfaces.
